SEQUENCE LISTING (37 sequences)

SEQ ID NO: 1 - protein, 32 aa, Artificial Sequence
  Description of Artificial Sequence Consensus sequence
LXXXXXXLXXXXXXLXXXXXXLXXXXXXLXXX

SEQ ID NO: 2 - protein, 31 aa, Artificial Sequence
  Description of Artificial Sequence Synthetic peptide
LEEKVKTLKAQNYELASTANMLREQVAQLKQ

SEQ ID NO: 3 - protein, 31 aa, Artificial Sequence
  Description of Artificial Sequence Synthetic peptide
LKEKVKTLKAQNYELASTANMLREQVAQLKQ

SEQ ID NO: 4 - protein, 31 aa, Artificial Sequence
  Description of Artificial Sequence Synthetic peptide
LEEEVKTLEAQNYELASTANMLREQVAQLKQ

SEQ ID NO: 5 - protein, 31 aa, Artificial Sequence
  Description of Artificial Sequence Synthetic peptide
LEEKVKTLKAQNYELASTANMLEEEVAQLKQ

SEQ ID NO: 6 - protein, 31 aa, Artificial Sequence
  Description of Artificial Sequence Synthetic peptide
LEEEVKTLEAQNYELASTANMLEEEVAQLKQ

SEQ ID NO: 7 - protein, 31 aa, Artificial Sequence
  Description of Artificial Sequence Synthetic peptide
LKEKVKTLEAQNYELASTANMLREQVAQLKQ

SEQ ID NO: 8 - protein, 31 aa, Artificial Sequence
  Description of Artificial Sequence Synthetic peptide
LKEEVKTLEAQNYELASTANMLREQVAQLKQ

SEQ ID NO: 9 - protein, 31 aa, Artificial Sequence
  Description of Artificial Sequence Synthetic peptide
LKEKVKTLKAQNYELASTANMLEEEVAQLKQ

SEQ ID NO: 10 - protein, 31 aa, Artificial Sequence
  Description of Artificial Sequence Synthetic peptide
LKEEVKTLEAQNYELASTANMLEEEVAQLKQ

SEQ ID NO: 11 - protein, 31 aa, Artificial Sequence
  Description of Artificial Sequence Synthetic peptide
LEEKVKTLKAQNYELASTANMLREQVAQLEQ

SEQ ID NO: 12 - protein, 31 aa, Artificial Sequence
  Description of Artificial Sequence Synthetic peptide
LKEKVKTLKAQNYELASTANMLREQVAQLEQ

SEQ ID NO: 13 - protein, 31 aa, Artificial Sequence
  Description of Artificial Sequence Synthetic peptide
LEEKVKTLEAQNYELASTANMLREQVAQLEQ

SEQ ID NO: 14 - protein, 31 aa, Artificial Sequence
  Description of Artificial Sequence Synthetic peptide
LEEEVKTLEAQNYELASTANMLREQVAQLEQ

SEQ ID NO: 15 - protein, 31 aa, Artificial Sequence
  Description of Artificial Sequence Synthetic peptide
LEEKVKTLKAQNYELASTANMLEEEVAQLEQ

SEQ ID NO: 16 - protein, 31 aa, Artificial Sequence
  Description of Artificial Sequence Synthetic peptide
LEEEVKTLEAQNYELASTANMLEEEVAQLEQ

SEQ ID NO: 17 - protein, 31 aa, Artificial Sequence
  Description of Artificial Sequence Synthetic peptide
LEKKAEDLSSLNGQLQSEVTLLRNEVAQLKQ

SEQ ID NO: 18 - protein, 31 aa, Artificial Sequence
  Description of Artificial Sequence Synthetic peptide
LKKKAEDLSSLNGQLQSEVTLLRNEVAQLKQ

SEQ ID NO: 19 - protein, 31 aa, Artificial Sequence
  Description of Artificial Sequence Synthetic peptide
LEKKAEDLSSRNGQLQSVVTLLRNEVAQLKQ

SEQ ID NO: 20 - protein, 31 aa, Artificial Sequence
  Description of Artificial Sequence Synthetic peptide
LKKKAEDLSSRNGQLQSVVTLLRNEVAQLKQ

SEQ ID NO: 21 - protein, 31 aa, Artificial Sequence
  Description of Artificial Sequence Synthetic peptide
LKXKXXXLKXQXXXLAXTXXXLRXQXXXLKX

SEQ ID NO: 22 - protein, 31 aa, Artificial Sequence
  Description of Artificial Sequence Synthetic peptide
LEXEXXXLEXQXXXLAXTXXXLRXQXXXLKX

SEQ ID NO: 23 - protein, 31 aa, Artificial Sequence
  Description of Artificial Sequence Synthetic peptide
LEXKXXXLKXQXXXLAXTXXXLEXEXXXLKX

SEQ ID NO: 24 - protein, 31 aa, Artificial Sequence
  Description of Artificial Sequence Synthetic peptide
LEXEXXXLEXQXXXLAXTXXXLEXEXXXLKX

SEQ ID NO: 25 - protein, 31 aa, Artificial Sequence
  Description of Artificial Sequence Synthetic peptide
LKXKXXXLEXQXXXLAXTXXXLRXQXXXLKX

SEQ ID NO: 26 - protein, 31 aa, Artificial Sequence
  Description of Artificial Sequence Synthetic peptide
LKXEXXXLEXQXXXLAXTXXXLRXQXXXLKX

SEQ ID NO: 27 - protein, 31 aa, Artificial Sequence
  Description of Artificial Sequence Synthetic peptide
LKXKXXXLKXQXXXLAXTXXXLEXEXXXLKX

SEQ ID NO: 28 - protein, 31 aa, Artificial Sequence
  Description of Artificial Sequence Synthetic peptide
LKXEXXXLEXQXXXLAXTXXXLEXEXXXLKX

SEQ ID NO: 29 - protein, 31 aa, Artificial Sequence
  Description of Artificial Sequence Synthetic peptide
LEXKXXXLKXQXXXLAXTXXXLRXQXXXLEX

SEQ ID NO: 30 - protein, 31 aa, Artificial Sequence
  Description of Artificial Sequence Synthetic peptide
LKXKXXXLKXQXXXLAXTXXXLRXQXXXLEX

SEQ ID NO: 31 - protein, 31 aa, Artificial Sequence
  Description of Artificial Sequence Synthetic peptide
LEXKXXXLEXQXXXLAXTXXXLRXQXXXLEX

SEQ ID NO: 32 - protein, 30 aa, Artificial Sequence
  Description of Artificial Sequence Synthetic peptide
LEXEXXXLEXQXXXLAXTXXXLRXXXXLEX

SEQ ID NO: 33 - protein, 31 aa, Artificial Sequence
  Description of Artificial Sequence Synthetic peptide
LEXKXXXLKXQXXXLAXTXXXLEXEXXXLEX

SEQ ID NO: 34 - protein, 31 aa, Artificial Sequence
  Description of Artificial Sequence Synthetic peptide
LEXEXXXLEXQXXXLAXTXXXLEXEXXXLEX

SEQ ID NO: 35 - protein, 32 aa, Artificial Sequence
  Description of Artificial Sequence Synthetic peptide
LKXKXXXLSXLXXXLQXEXXXLRXEXXXLYKX

SEQ ID NO: 36 - protein, 31 aa, Artificial Sequence
  Description of Artificial Sequence Synthetic peptide
LEXKXXXLSXRXXXLQXVXXXLRXEXXXLKX

SEQ ID NO: 37 - protein, 31 aa, Artificial Sequence
  Description of Artificial Sequence Synthetic peptide
LKXKXXXLSXRXXXLQXVXXXLRXEXXXLKX

(19) United States
(12) Patent Application Publication
(10) Pub. No.: US 2002/0165153 A1
(43) Pub. Date: Nov. 7, 2002

(54) Transcription factors and their use

(76) Inventors: Peter Angel, Eggenstein-Leopoldshafen (DE); Peter Herrlich, Karlsruhe (DE); Hans Van Dam, Leiden (NL); Alex Van Der Eb, Oegstgeest (NL)

Correspondence Address: KLAUS J. BACH & ASSOCIATES, 4407 TWIN OAKS DRIVE, MURRYSVILLE, PA, 15668, US

(21) Appl. No.: 10/051,804

(22) Filed: Jan. 11, 2002

Related U.S. Application Data
(63) Continuation-in-part of application No. 09/679,695, filed on Oct. 5, 2000.
Application No. 09/679,695 is a continuation-in-part of application No. PCT/EP99/01805, filed on Mar. 18, 1999.

(30) Foreign Application Priority Data
Apr. 6, 1998 (DE) 190 15 331.7

(51) Int. Cl. A61K 38/47; C12N 9/22

(52) U.S. Cl. 514/12; 435/199

(57) ABSTRACT

The invention relates to transcription factors with a DNA binding domain consisting of a basic domain and an adjacent leucine zipper domain. The aim of the invention is to provide modified transcription factors whose binding behavior towards each other and towards DNA sequences can be manipulated. To this end, at least one amino acid on the leucine zipper is replaced by a charged amino acid.

This is acontinuation-in-part application of U.S. patent application Ser. No. 09/679,695 filed Oct. 5, 2000 which is a continuation-in-part application of international patent application PCT/EP99/01805 filed Mar. 18, 1999 and claiming the priority of German application 198 15 331.7 filed Apr. 6, 1998.

### BACKGROUND OF THE INVENTION

The present invention relates to transcription factors with a DNA binding domain comprising at least a basic domain and an adjacent leucine-zipper domain with the following amino acids:

L el x gl x x x L e2 x g2 x x x′x L e3 x g3 x x x L e4 x g4 x x x L e5

wherein L is leucine, gi and ei are possible substitution locations and x are any amino acids and x′ is tyrosin or glycin.

Van Straaten et al. and Angel et al. have described such transcription factors (van Straaten, F., Muller, R., Curran, T., van Beveren, C., and Verma, I. M. (1983) Proc. Natl. Acad. Sci. USA, 80, 3183-3187; Angel, P. Allegretto, E. A., Okino, S. T., Hattori, K., Bolyle, W. J., Hunter, T., and Karmi, M., (1988) Nature 332, 166-171). Vinson et al. describe the mechanism of the dimerization of these transcription factors with the aid of the zipper domains (Vinson, C. R., Hai, T., and Boyd, S. M. (1993) Genes Dev. 7, 1047-1058). It was postulated herein, that in the two dimerizing zipper domains the amino acid in the g position (three positions carboxyterminal of leucine) of the one dimer partner interacts with the amino acid being in the next e position (five positions carboxyterminal) of the other dimer partner. This postulation was called the (gi−ei+1) principle. By these (gi−ei+1) interactions, the specificity of dimerization of two leucine zipper domains would be determined. However, by testing the (gi−ei+1) principle for the leucine zipper containing transcription factors cJun and ATF2, we found that the principle cannot be transferred in a general way to the binding behavior of the complete proteins to DNA sequences.

It is the object of the present invention to provide modified transcription factors whose binding behavior relative to each other and to DNA sequences can be manipulated.

### SUMMARY OF THE INVENTION

The invention relates to transcription factors with a DNA binding domain consisting of a basic domain and an adjacent leucine zipper domain. The aim of the invention is to provide modified transcription factors whose binding behavior towards each other and towards DNA sequences can be manipulated. To this end, at least one amino acid on the leucine zipper is replaced by a charged amino acid.

A particular advantage of the transcription factors according to the invention resides in the fact that it is possible, by suitable substitution of amino acids, to select reaction partners in a predetermined way. It is especially possible to modify the binding behavior to DNA sequences by suitable substitutions, which was not possible via the (gi−ei+1) principle described above.

Below the invention will be described in greater detail on the basis of same embodiments utilizing a table:

### DESCRIPTION OF THE TABLE

The table shows a protein sequence in the area of the leucine zipper for various substituents with two different transcription factors cJun (Angel et al., 1988, see above) and ATF2 (Mackawa, T., Sakura, H., Kanei—Ishii, C., Sudo, T., Yoshimura, T., Fujisawa, J. -i., Yoshida, M., and Ishii, S. (1989) EMBO J. 8, 2023-2028).

The table shows, in the lines, the amino acid sequence (position 280-310 for 14 substituents of cJun and three of ATF2 (position 380-410). el to eS and g1-g4 are the possible substitution positions. The letters correspond to the usual amino acid code.

Description of the Substitutions:

A summary of the specific behavior of the various substituents is presented. Part of the experiments are described in detail in the two publications added as appendix (van Dam, H., Huguier, S., Kooistra. K., Baguet, J., Vial, E., van der Eb, A. J., Herrlich, P., Angel, P. and Castellazzi, M. (1998) Genes Dev., 12, 1998, 1127-1239; Huguier, S., Baguet, J., Perez, S., van Dam, H., and Castellazzi, M. (1998). Mol. Cell. Biol. 18, 7020-7029). Further experimental details are available upon request.

cJun-m0:

1) has, without DNA, in comparison with wild-type cJun an increased affinity for the cJun dimer partner cFos (van Straaten et al., 1983, see above).

2) binds well to DNA when it dimerizes with cFos, but badly when it is offered with ATF2; it binds very badly as Jun-m0/Jun-m0 homo-dimer.

3) in comparison with wild-type cJun, cJun m0 activates various Jun/Fos-dependent genes, including the 5xcollTRE-tata-luciferase reporter gene, relatively efficiently in several cell types, but Jun/ATF-dependent genes, including the Jun/ATF2-dependent reporter gene 5xjun2-tata-luciferase, only very inefficiently.

4) it does not bind to DNA with ATF2-m5 (see below), it does not bind to DMA with wt cJun or JunD (Hirai; S. J., Rysek, R. -P., Mechta, F., Bravo, R., and Yaniv, M. (1989) EMBO J.8, 1433-1439).

(Described extensively in: van Dam et al., 1998; Huguier et al., 1998; see appendix.) cJun-m35:

1) without DNA, it binds in comparison with wt cJun somewhat better to ATF2 than to cFos

2) it binds in comparison with wt Jun homodimers much stronger to DNA as Jun-m35/Jun-m35 homodimers or wtJun/Jun-m35 heterodimers; it also binds well to DNA as Jun/ATF2, the binding to DNA as Jun/Fos is relatively weak.

3) in comparison with wt cJun it activates the Jun/Fos dependent reporter to a much smaller degree and the Jun/ATF2 dependent promoter to a higher degree.

4) it binds very well to DNA with ATF2-m5(see below)

5) it binds relatively well to DNA with JunD.

cJun-m2

1) without DNA, it binds relatively badly to ATF2 but relatively well to cFos,

2) without DNA, it binds to JunD very badly or not at all,

3) it binds badly to DNA with wt cJun and with JunD,

4) it binds badly to DNA with ATF2-m5 (see below)

cJun-ml:

1) without DNA, it binds, in comparison with wt Jun, much weaker to cFos, but well with ATF2,

2) it binds well to DNA when dimerized with ATF2 but badly when offered together with cFos

3) in comparison with the wild-type cJun it activates in several cell types the Jun/Fos-dependent reporter-gene 5xcollTRE-tata-luciferase and other Jun/Fos-dependent genes very inefficiently, but the Jun ATF2-dependent reporter gene 5xjun2-tata-luciferase and other Jun/ATF-dependent genes very well.

4) it binds very well to DNA with ATF2-m5 (see below)

5) without DNA, it binds relatively strongly to wt cJun and JunD.

(Described extensively in: van Dam et al., 1998; Huguier et al., 1998; see appendix.)

cJun-m0,3:

1) it binds badly to DNA with ATF2 and as homodimer,

2) it binds badly to DNA with ATF2-m5 (see below)

cJun-m0,35:

1) it differs from cJun-m35 (see above) by a weaker binding to DNA than homodimer,

2) it has a relatively weak binding to DNA with wtcJun and Jun D,

3) it differs from the cJun-m35 by a weak binding to DNA with ATF2-m5.

cJun-m0,1:

1) in comparison with wt Jun, its binding to cFos without DNA is weak, but its binding to ATF2 is good,

2) it binds well to DNA when dimerized with ATF2, but relatively weakly if offered together with cFos since it dimerized badly with cFos.

3) in comparison with the wild-type cJun, it activates, in several cell types, the Jun/Fos dependent reporter gene 5xcollTRE-tata-luciferase inefficiently, but the June/ATF2 dependent reporter gene 5xjun2-tata-luciferase very well.

4) in comparison with ml (see above), it binds relatively weakly to wt cJun and JunD.

5) it binds weakly to DNA with ATF2-m5.

cJun-m5:

1) without DNA, it binds relatively badly to ATF2, but well to cFos,

2) it binds relatively strongly to DNA with Fos and as a m5/m5 homodimer,

3) without DNA, it binds very weakly to JunD.

cJun m0,5:

1) As a homodimer, it binds very weakly to DNA,

2) it binds very badly to DNA with ATF2-m5

cJun-m3,5:

1) it binds very strongly to DNA as a homodimer,

2) it binds very badly to DNA when dimerized with ATF2-m5 (see below).

cJun-m35,5:

1) it binds very strongly to a DNA as a homodimer,

2) it binds relatively strongly to DNA with ATF2-m5 (see below).

cJun-m2.5:

1) without DNA, it binds weakly to ATF2 but well to cFos,

2) it efficiently activates Jun/Fos-dependent reporter genes, but not Jun/ATF2-dependent genes

3) without DNA, it binds weakly to JunD.

cJun-m1.5:

1) without DNA, it binds badly to cFos and relatively well to ATF2.

2) it does not or very badly bind to DNA as Jun/Fos and weakly as Jun/ATF2.

3) it does not significantly activate Jun/Fos-dependent reporter genes, but does activate Jun/ATF2-dependent genes.

4) without DNA, it binds strongly to JunD.

5) it binds weakly to DNA with ATF2-m5 (see below).

ATF2-ml:

1) it binds weakly to DNA as a homodimer and as a heterodimer to cJun-wt,

2) it binds well to DNA with cJun-m35 (see above).

ATF2-m2:

1) it binds weakly to DNA as a homodimer but, in contrast to ATF2-ml and m5 (see below), it binds relatively well with wt cJun.

2) it binds weakly to DNA with cJun-m35 (see above)

ATF2-m5

1) in comparison with ATF2, it binds relatively weakly on wt cJun without DNA.

2) it does not bind to DNA as a homodimer or as a hetero dimer with wt cJun.

3) it binds well to DNA with cJun-m35 (see above)

4) it binds weakly to DNA as ATF2/cFos (wt ATF2 does not bind to DNA with cFos).

Methods Used for the Characterization of the Mutants:

a) Protein-interaction without DNA was measured using a so-called “mammalian two hybrid analysis” for which the mutant zipper was fused with the DNA binding domain of the yeast protein GAL4(GAL4-D). The interaction with cFos, ATF2 or JunD was quantified by the introduction of one of these proteins with the cJun-GAL4-D fusion protein into the same cell together with a GAL4-dependent reporter gene. The activation of the reporter construct is a measure for the protein-protein interaction.

b) DNA binding as Jun/Jun and Jun/Fos dimers was measured to a 20 base pair long double-stranded oligonucleotide with the core motive TGAGTCA as essential DNA sequence. For the DNA binding as Jun/ATF2 or ATF2 dimers a similar oligonucleotide with core motive TTACCTCA was used.

(Described extensively in: van Dam et al., 1998; Huguier et al., 1998; see appendix.)

Examples for the Deactivation and Hyperactivation of Transcription Factors

1. Deactivation by deletion or mutation of the complete or of certain parts of the transactivation domain:

a) deletion of the complete transactivation domain of cJun (amino acid 6 to 194) or ATF2 (amino acid 1-110 of ATF2).

b) deletion of individual subdomains of the transactivation domain of cJun (region I, II and/or III; HOB domain; delta domain as docking location for MAP kinases.

c) mutation of the phosphorylation locations Ser63 and/or Ser 73, Thr89, Thr91, Thr93 of cJun or Thr69, Thr71 and Ser90 of ATF2: substitution by an Ala or Leu amino acid.

d) deletion of the zinc-finger domain in the amino terminal part of ATF2; deletion of the binding domain in ATF2 for co-activators. See for instance; Angel, P., Smeal, T., Meek, J., and Karin, M. (1989). New Biol. 1, 35-43.; Flint K J and Jones N C. (1991). Oncogene 6, 2019-2026).

2. Deactivation by deletion or mutation of parts of the DNA binding domain (for example, basic domain: blocking of the DNA binding, only dimerization is still possible).

3. Hyperactivation: by changes on the transactivation domain:

a) substitution of Ser63 and/or Ser73, Thr89, 91, 93 of cJun and Thr69 and/or Thr71, Ser 90 of ATF2 by Asp.

b) Fusion of cJun or ATF2 with the hyperactive transactivation domain of the Herpes Simplex virus VP16 protein.

Examples for the Use as Therapeutica:

a) The ratio of cJun-Fos activity and cJun-ATF2 activity plays an important role in DNA-damage-induced apoptosis and other types of programmed cell death (Schreiber M, Baumann B, Cotten M, Angel P, Wagner E F (1995) EMBO J.14, 5338-5349; van Dam H, Wilhelm D, Herr I, Steffen A, Herrlich P, Angel P. (1995) EMBO J. 14, 1798-1811; Kolbus A, Herr I, Schreiber M, Debatin K M, Wagner E F, Angel P. (2000) Mol. Cell Biol 20, 575-582). Stable or transient introduction of cJun-m0 in immortalized mouse fibroblasts and neuronal cells only efficiently upregulates Jun/Fos-dependent gene expression and makes these cell less sensitive to apoptosis induced by growth factor-depletion. In contrast, introduction of cJun ml or cJun m0,1 only efficiently upregulates Jun/ATF-dependent gene expression and makes the same cells more sensitive to apoptosis induced by growth factor-depletion. Moreover, cJun-ml also makes these cells more sensitive to apoptosis induced by alkylating drugs (H. van Dam et al., unpublished results). Combinations of normal or hyperactive ATF2-m5 or of ATF2-ml together with normal or hyperactive cJun-ml, m0.1, m35 or m1.5 mutants are even more effective in activating Jun/ATF-dependent gene expression in certain cell types (van Dam et al., 1998; Huguier et al., 1998; see appendix.)

The ATF-preferring mutants are therefore potential therapeutics for the treatment of cancer types wherein cell death can be caused by activating cJun-ATF2 activity (for example, cancer cells of the ovary which are sensitive to Cisplatin). Moreover, in cells of patients in which undesired (programmed) cell death occurs, cJun-m0 and cJunm2,5 (or their hyperactive derivatives) consequently can influence the balance of Jun/Fos activity in comparison with Jun/ATF2 activity and potentially prevent undesired cell death. These versions of m0 and m2,5 are consequently suitable therapeutics to inhibit such cases of undesired cell death.

b) Upon introduction in chicken cells with the use of a retroviral vector, Jun-m1, Junm0,1 and Jun-mO activate complementary carcinogenic programs: Growth factor-independency and primary tumor formation is induced by Jun-m1 and Jun-m0.1 whereas growth in a semi-solid medium, which is related to metastasis formation, is induced by Jun-m0 (Described extensively in: van Dam et al., 1998; Huguier et al., 1998; see appendix.) Moreover, metastasis of various types of cancer cells is induced or increased by the activation of Jun/Fos-activity for example by the induction of genes which code for cell surface proteins like the CD44 protein. As inactive versions of Jun-m1 and Jun-m0 lacking the Jun transactivation domain can inhibit cell growth, these mutants are potential inhibitors of primary tumor formation and metastasis.

Proposed method of introducing the mutant transcription factors in vivo:

a) as gene construct via gene therapy: for example, in an Adeno virus vector or in a retro-viral vector (Verma, 27, and Somia, N. (1997) Native 389, 239-242; Mulligan, R. C. (1993) Science 260, 926-932. Already described for chicken cells in: van Dam et al., 1998; Huguier et al., 1998; see appendix.)

b) as small peptide analog to the MdM2/p53 peptides.

Examples of Test Systems for Medications:

As described above, normal, hyperactive or (partially) deactivated forms of cJun-m0, or m1, m0.1, m35, m1,5 and m2,5 differ in their capacity to form Jun/Fos, Jun/Jun and Jun/ATF2 diners and to switch on Jun/Fos or Jun/ATF2 dependent reporter genes. This offers the possibility to specifically examine the roles of individual Jun-dimers in Jun-controlled biological processes like programmed or other types of cell deaths, cell survival, different types of cancer, embryonal development, excessive stress responses and cell differentiation. In addition, these mutants offer the possibility to examine corresponding medications for their specificity with respect to the various types of Jun-dimers and the associated biological processes. For instance, medications can be tested which inhibit certain specific aspects of cancer growth, for example, metastasis. Also, medications can be tested which are to inhibit or increase Jun/Fos and Jun/ATF2 induced cell death.

Proposed Test System for the Testing of Medications:

In vitro cell cultures and transgenic mice in which the mutant transcription factors are expressed by standard gene technology.

Modeling of nuclear receptors

In addition to the main role as transcription factors the Jun-dimers are effective also in the interaction with other transcription factors. In many cases, this interaction results in mutual inhibition, for example, the glucocorticoid-receptor inhibits the activity of Jun/Fos on Jun/Fos target genes.

Vice versa Jun/Fos interfers with the gene-activating effect of the glucocorticoid-receptors. This type of the transcription factor effect is the basis of the inflammation inhibition by cortisol, the cancer inhibiting effect of the retinoids and they are vital for the organism. The complex interactions of the mutants, and peptides according to the invention with nuclear receptors can be used to influence this interaction with the methods mentioned above. In addition, for example the Jun-mutants can be utilized for establishing a screening test for inflammation-inhibiting and anti-tumor ligands of the nuclear receptors.

General Use of Mutant Transcription Factors as Medications or for the Testing of Medications:

The specificity mechanism can be transferred to other proteins which include bZIP (basic domain leucine zipper) and to proteins which interreact by way of a Leucine-zipper. Examples are JunB, Jun D, ATFa, ATF3, CREB, ATF1, MAF, CEBP (Hurst, H. C. 1994, Transcription factors: 1: bZIP proteins. Protein profile 1, 123-168). Therefore the normal zippers of these bZIP proteins can be replaced by one of the above-mentioned zippers and the role, in biological processes, of individual dimers, which are formed for example by JunD or ATF3, can be examined as described above for Jun/Fos and Jun/ATF2. Deactivated and hyperactive versions of these other bZIP transcription factors can be utilized in this manner to intervene therapeutically in several types of diseases, in which bZIP transcription factors play a role, such as auto-immune diseases, cancer, genetic deviations and embryonal development problems.

As already described above, also medications can be tested with these mutants which are specific with respect to the individual ATF3, ATFa, JunD, CEBP etc., proteins may play a role can be influenced in this way.

General use of Leucine Zipper Domain Mutants for Specific Intervening in Protein-Protein Interaction in vitro and in vivo

A large amount of proteins interact with other proteins by way of their leucine zipper domain. Therefore the above mentioned zipper domains can be used with changed interaction specificity to intervene selectively in intersections of all proteins including a Leucine zipper. The normal zipper domain is replaced by a suitable one of the above described mutant zippers as for example Jun-m0, ATF2-m5 or cJun-m1,5. Furthermore, proteins can be coupled in a controlled way by the elimination with gene technology of other types of protein-interaction domains and by the introduction of mutant zippers as described above.

Mutant Zippers as Coupling Partners of Various Action Groups.

The zipper domain of cJun-m0 has at a neutral pH a strongly positive charge because cJun-m1 and cJun-m1.5 have a strongly negative charge. Therefore m0 on one hand and m1 on the other, are very suitable as coupling partners of bio molecules.

With the aid of the usual genetic and (bio)chemical methods, the m0, m1 and m1.5 polypeptides can be coupled to other effective components. These mutant domains are for example well suited for use in drug-targeting with certain cell types. It is possible to fuse certain biologically active proteins by way of gene technology with the m0 zipper and to introduce them into the body. The cell components to be targeted are also marked with the m1.5 zipper.

TABLE

| | e1 | g1 | e2 | g2 | e3 | g3 | e4 | g4 | e5 |
|---|---|---|---|---|---|---|---|---|---|
| cJun wt | LEEKVKTLKA Q NYELASTANMLREQVAQLKQ | | | | | | | | |
| cJunwt | E | K | K | Q | A | T | R | Q | K (SEQ ID NO 2) |
| m0 | K | K | K | Q | A | T | R | Q | K (SEQ ID NO 3) |
| m35 | E | E | E | Q | A | T | R | Q | K (SEQ ID NO 4) |
| m2 | E | K | K | Q | A | T | E | E | K (SEQ ID NO 5) |
| m1 | E | E | E | Q | A | T | E | E | K (SEQ ID NO 6) |
| m0,3 | K | K | E | Q | A | T | R | Q | K (SEQ ID NO 7) |
| m0,35 | K | E | E | Q | A | T | R | Q | K (SEQ ID NO 8) |
| m0,2 | K | K | K | Q | A | T | E | E | K (SEQ ID NO 9) |
| m0,1 | K | E | E | Q | A | T | E | E | K (SEQ ID NO 10) |
| m5 | E | K | K | Q | A | T | R | Q | E (SEQ ID NO 11) |
| m0,5 | K | K | K | Q | A | T | R | Q | E (SEQ ID NO 12) |
| m3,5 | E | K | E | Q | A | T | R | Q | E (SEQ ID NO 13) |
| m35,5 | E | E | E | Q | A | T | R | Q | E (SEQ ID NO 14) |
| m2,5 | E | K | K | Q | A | T | E | E | E (SEQ ID NO 15) |
| m1,5 | E | E | E | Q | A | T | E | E | E (SEQ ID NO 16) |

ATF2 wt LEKKAEDLSSLNGQLQS EVTL LRNEVAQLKQ

| | | | | | | | | | |
|---|---|---|---|---|---|---|---|---|---|
| ATF2 wt | E | K | S | L | Q | E | R | E | K (SEQ ID NO 17) |
| m1 | K | K | S | L | Q | E | R | E | K (SEQ ID NO 18) |
| m2 | E | K | S | R | Q | V | R | E | K (SEQ ID NO 19) |
| m5 | K | K | S | R | Q | V | R | E | K (SEQ ID NO 20) |

What is claimed is:

1. A transcription factor with a DNA-binding domain comprising a basic domain and an adjacent Leucine zipper domain with the following amino acids:
L el x gl x x x L e2 x g2 x x x′ x L e3 x g3 x x x L e4 x g4 x x x L e5
wherein L is leucine, gi and ei are possible substitution locations, x are any amino acids, and x′ is one of tyrosin and glycin and wherein one of the following sets of substituents are at the nine substitution locations ei and gi:

| | |
|---|---|
| KK KQ AT RQ K | SEQ ID NO 3 |
| EE EQ AT RQ K | SEQ ID NO 4 |
| EK KQ AT EE K | SEQ ID NO 5 |
| EE EQ AT EE K | SEQ ID NO 6 |
| KK EQ AT RQ K | SEQ ID NO 7 |
| KE EQ AT RQ K | SEQ ID NO 8 |
| KK KQ AT EE K | SEQ ID NO 9 |
| KE EQ AT EE K | SEQ ID NO 10 |
| EK KQ AT RQ E | SEQ ID NO 11 |
| KK KQ AT RQ E | SEQ ID NO 12 |
| EK EQ AT RQ E | SEQ ID NO 13 |
| EE EQ AT RQ E | SEQ ID NO 14 |
| EK KQ AT EE E | SEQ ID NO 15 |
| EE EQ AT EE E | SEQ ID NO 16 |
| KK SL QE RE K | SEQ ID NO 18 |
| EK SR QV RE K | SEQ ID NO 19 |
| KK SR QV RE K | SEQ ID NO 20, |

valin being replaceable by alanine, leucine, methionine, isoleucine, glutamine.

2. The transcription factor according to claim 1, wherein the transactivation domain is either deactivated or hyperactivated.

3. The transcription factor according to claim 1, for the manufacture of a therapeuticum.

4. The transcription factor according to claim 1, for the testing of indications.

5. The transcription factor according to claim 1, for the coupling of effective groups including biomolecules.
